(19) United States
(12) Patent Application Publication
(10) Pub. No.: US 2010/0119539 A1
(43) Pub. Date: May 13, 2010

(54) VACCINE AGAINST MALARIA, BASED ON THE 200L SUBUNITI OF PLASMODIUM VIVAX MSP1 PROTEIN

(75) Inventors: Augusto Elías Valderama Aguirre, Cali (CO); Socrates Herrera Valencia, Cali (CO); Myriam Arévalo Ramirez, Cali (CO); David Narum, Rockville, MD (US)

Correspondence Address: MERCHANT & GOULD PC, P.O. BOX 2903, MINNEAPOLIS, MN, 55402-0903, US

(73) Assignees: Centro Internacional De Vacunas, Barrio San Fernando (CO); Instituto Inmunologia Universidad Del Valle, San Fernando (CO)

(21) Appl. No.: 12/514,910

(22) PCT Filed: Nov. 14, 2006
(86) PCT No.: PCT/IB06/03835

§ 371 Date: Jan. 15, 2010

(51) Int. Cl. A61K 39/015; C12N 15/09; C12N 1/21; C12N 15/63; C07K 2/00; C12N 15/11

(52) U.S. Cl. 424/191.1; 435/69.3; 435/252.33; 435/320.1; 530/350; 536/23.1

(57) ABSTRACT

A candidate subunit for a vaccine against malaria caused by P. vivax, known as Pv200L, which is based on N-terminal end portions of the P. vivax MSP-1 protein is disclosed. The subunit is designed for use alone or in formulations, combined with other subunits. The production of recombinant prototypes of the subunit and the design of a production process that can be scaled up for mass production thereof is also disclosed.

### INVENTION FIELD

The invention described below is referred to vaccines against malaria, based on the 200L subunit, comprising aminoacids between 50 and 450 residues of the P. vivax Merozoite Surface Protein 1 (MSP1) and are aimed to control the development of blood stages and the disease severity.

### INVENTION BACKGROUND

Malaria is one of the major public health problems worldwide. An estimated of 500 million clinical cases are produced annually around the world and only in Africa almost 3 million children and pregnant women die by this disease every year

Africa is the most affected continent by malaria, particularly by Plasmodium falciparum, the most virulence species and responsible of around 80% of malaria worldwide. The second species in abundance is P. vivax, which represents almost 20% of cases and is significantly transmitted in the America and Asia continents In these two continents the majority of endemic regions have simultaneous transmission of P. falciparum and P. vivax. In many malaria regions, the prevalence of P. vivax is higher, although it does not cause mortality its produces a significant morbidity.

P. vivax is characterized because produced a debilitating and high incapacitating disease that presents a recidivate behavior if the infection is not adequately treated. This last characteristic represents a high risk for tourist and travelers infected for first time because they can develop repetitive infections without exposition of new mosquito bites.

Malaria has a negative impact on social and economic development in endemic areas producing during last 20 years, a reduction of about 50% of the accumulated Gross Domestic Product (GDP) in malaria endemic countries as compared with non-endemic countries.

Currently, the costs of control management and treatment of malaria are excessively high. The World Health Organization (WHO) has estimated that the annual cost for malaria prevention would be around US$2.5 billion for 2007 only in Africa.

Since several decades, World Health Organization has recommended classic control methods which consist of two alternatives:
1. Vector control by elimination of their source, the use of repellent and impregnate bednets and elimination by insecticide of residual action
2. Preventive and healing treatment on malaria exposed or infected individuals by the used of antimalarial drugs.

Classical measures of malaria control have been failed due to parasite resistance to antimalarial therapy and resistance of Anopheles mosquitoes to insecticides, resulting in an increased complexity and high cost of malaria control around the world. This factor together with deforestation, migrations and politic instability of communities in endemic areas contributes to exacerbate the problem.

During the past two decades, significant efforts have been made to develop alternative strategies for control of its transmission, in which the vaccines have attracted increased interest for its potential cost-benefit

To develop a malaria vaccines it is required to understand the Plasmodium life cycle, and define what is common to the four species of this genus (Plasmodium spp) that affects humans (P. falciparum, P. vivax, P. malaria, and P. ovale) and which are the molecules involved in it maintenance.

In the life cycle, the parasite is transmitted from one infected subject to another by Anopheles spp mosquitoes bites, injecting sporozoites into the human blood. Parasites travel from the blood to the liver where they invade the liver cells and develop a phase of mass multiplication (Schizogonic cycle) generating millions of new parasites (merozoites) able to invade red blood cells, within which the parasite develops a succession of cycles of multiplication and reinvasion of new red blood cells, increasing rapidly in number in the body.

This latest series of events in the blood are responsible for the disease and can lead to death. Some of the parasites in the blood differentiate into sexual gametocytes (male and Female) that after ingestion by the mosquito during a new bite, do a process of fertilization and initiate a new cycle (sporogonic or sexual) in the gut of the mosquito, to generate new infectious sporozoites.

During the erythrocytic stage of P. vivax merozoite from the liver origin, invades a young red blood cell or reticulocytes and there is multiplied by asexual mechanisms to generate mature schizonts containing 20-30 new merozoites (Schizogony). Merozoites escape from schizont and invade new red cells which in turn are faster to generate new merozoites. Some of the parasites in the liver may stop its development and transformed into hibernating forms (hipnozoites) which can reactivate months or years later and developed the disease after a first episode if treatment is not suitable.

Within the complex life cycle of Plasmodium, three target stages has been identified to assay an antimalarial vaccine: 1) in the pre-erythrocytic stage, which is before the entry of the parasite in the liver or it development; 2) in the asexual erythrocytic stage, which is before or during red cells invasion, or in their intra-erythrocytic development and 3) during their sexual development, fertilization and into the mosquito gut development. Of the three targets mentioned only the erythrocytic phase is responsible for the symptoms and the severity of the infection. The pre-erythrocytic stage occurs without symptomatology and the sexual reproduction take place into the mosquito.

Asexual erythrocytic vaccines are based on “premonition” phenomena. Habitants of endemic areas exposed to successive infections, acquire protective immunity against clinic manifestations but not against the infection. As a result, subjects from these regions become “immune” and have a normal life. This immunity begins since early ages and is conserved until adult life by means of repeated infections that work as boosters of the immunologic memory. This type of partial immunity, non-sterile, is known as “Clinical Immunity” and is initially manifested with a reduced risk of death, then a reduction in the intensity or severity of clinical symptoms, and finally after many re-infections with a decrease in parasite load.

This phenomenon has been difficult to prove in P. vivax in low transmission areas, where habitants reach relatively low levels of clinical immunity because easily lost the frequency of natural boosting. In areas where are levels of moderate or high transmission as Papua New Guinea has been determined that the acute symptoms may reduce their intensity after a number of clinical episodes and the prevalence of infection declined significantly after the age of 5-10 years, reaching significant levels of clinical immunity at the age of 10-15 years

In the erythrocytic stage immunity is mediated primarily by antibodies and is considered that cellular immunity is limited because the red cell does not express a Major Histocompatibility Complex (MHC) molecule that allows them to present antigens. Studies of passive antibody transference have demonstrated to confer protective immunity and be able to control patent parasitemias in affected patients by P. falciparum. The mechanisms involved are: 1) locking of parasite invasion to red cells; 2) Parasite opsonization and agglutination with subsequent promotion of fagocitosis; 3) Antibody-dependent cellular inhibition; 4) Inhibition by sequestration in microcirculation; and 5) Neutralization of malarial toxins.

Despite being the only evidence of protective immunity induced by natural means, people in endemic areas are still affected by the disease. This is partly because immunological memory in malaria, for unknown reasons, is very short lasting. Prospective studies, have reported that if there is a period up to 6 months between two infections in the same subject, immunologic memory is lost and immune system responds as if it had never been in contact with the parasite. Therefore, the existence of a vaccine that could generate immunologic memory with long lasting against blood stages would be very useful, especially for people in endemic areas.

The Merozoite surface protein 1 (MSP1) is an antigen which is considered the first target of immune response against blood stages of Plasmodium spp. This antigen has been able to induce a partial or sterile protective immunity in several animal models. Experiments of passive transfer of antibody against different fragments of the protein had blocked the invasion process to red cells and induce partial protective immunity in animal models. Several subunits vaccine candidates against P. falciparum malaria have been developed from this antigen.

The MSP1 is abundantly expressed on the merozoite surface during the schizont maturation, is essential for normal development of schizont and for the initial interaction with the erythrocyte. During the rupture of schizont and the subsequent invasion to red cells, only one fragment of 19 KDa, localized toward the C-terminal extreme, remains anchored to the merozoite membrane. This fragment has similar domains to those of the Epidermal Growing Factor (EGF) that have been involved as ligands in the initial interaction of the erythrocyte, however, there are domains of attachment to red blood cells in other regions of the molecule. Toward the N-terminal region of the molecule, there is a highly conserved fragment with an unusual number of B and T epitopes that in P. falciparum has been denominated Pf190L. This fragment, expressed as a recombinant protein, can induce partial protective immunity against infectious challenge in primates (Herrera, Rosero et al. 1992), and has been included in a multivalent vaccine: “Combination B”, that has demonstrated to induce partial protection in clinical trials, being the 190L subunit, the most immunogenic (Saul, Lawrence et al. 1999).

Immunoepidemiological studies in Brazilian endemic areas have demonstrated the presence of antibodies and immune cells naturally induced against various regions of P. vivax MSP1. As in P. falciparum MSP1, attention has focused on the C-terminal region and subunits of 42 and 19 KDa fragments are well defined. Although it has been demonstrated that the N-terminal region has the highest immunogenicity in the molecule and that there is an association with clinic protection naturally induced, there are few studies leading to define an antigen subunit toward that side of the protein.

So far there are no patent applications that are related to a vaccine subunit from the P. vivax MSP-1 N-terminal region. Granted patents and/or in process that is referred to the MSP-1 antigen are usually directed to the C-terminal region of the P. falciparum protein, specifically to 42 and 19 KDa domains. (WO97/30150, US2004/0063190A1, WO02/085947A1, US2002/0076403, US2005/0095256A1) and the similar domains of the EGF (WO93/17107), none of the sequences previously mentioned have proven to have effects on the control of P. vivax infection.

Given the information above is clear that there is in the state of the technical art, the necessity of obtaining new antigens that allow, due to their immunogenic characteristics, the development of a vaccine against malaria for P. vivax.

### FIGURES DESCRIPTION

FIG. 1. Production of rPv200L

FIG. 2. Scalable production process of EcPv200L under GMP conditions

FIG. 3. Seroepidemiology of rPv200L in the Colombian Pacific Coast

FIG. 4. Immunogenicity of rPv200L in BALB/c mice

FIG. 5. Immunogenicity and protective efficacy of rPv200L in Aotus primates

### DETAILED DESCRIPTION OF THE INVENTION

The present invention is centered in the discovery, development and production of a target subunit vaccine, denominated rPv200L, which due to its immunogenic capability is consolidated as a candidate for the development of vaccines against P. vivax malaria.

Specifically, the subunit disclosed in this application is aimed to control parasitemia and infectious process severity during erythrocytic phase, in which the invasion of parasite (merozoite) to the young red cells (reticulocytes) occurs for the interaction of molecules (ligands) of the parasite surface and molecules (receptors) present in the reticulocyte surface. Development and intracellular multiplication can result blocked trough the action of cytokines induced by the protein, particularly interferon gamma (IFN-γ).

The invention includes the generation of vaccines, based on the subunit rPv200L, which aminoacid sequence and immunological significance was established by the applicant group (Valderrama-Aguirre, Quintero et al. 2005). The invention also includes the generation of the subunit by recombinant technology, together with the design and establishment of a process of generation and industrialized purification for the mass scale production of the subunit.

The present invention provides a unique approach for the production of a malaria vaccine candidate since it is based on the subunit Pv200L, which had not previously been described as a vaccine and less. Additionally, the invention is novel because it makes use of recombinant DNA technology and genetic engineering tools to modify the natural sequence of the generic fragment encoding the subunit Pv200L and thus increasing the efficiency of the synthesis and facilitating the subsequent E. coli purification process.

The subunit 200L is located toward N-terminal extreme of the P. vivax protein MSP-1 and was defined after bioinformatics analysis in which was determined a region of homology, higher than 70% with the subunit 190L of P. falciparum MSP-1 (Guttinger, Romagnoli et al. 1991), well defined candidate vaccine and in advanced clinical assessment. (Genton, Al-Yaman et al. 2000; Genton, Al-Yaman et al. 2003), followed by a region of high binding capacity to reticulocytes denominated HBRI (Rodriguez, Urquiza et al. 2002). The subunit is composed of part of block 1, blocks 2-4 and part of block 5 according to the classification of blocks by Putaporntip and collaborators (Putaporntip, Jongwutiwes et al. 2002) and is between 50-450 amino acids in most sequences of MSP-1 PV described to date.

In general, the subunit presents variations in the sequence and size of some of the blocks that compose it. For this reason, the invention unit is defined as a Pv200L consensus sequences (SEQ ID N^{o} 1) active in protection against P. vivax and obtained from variations observed in nature and punctual variations performed by the applicant to improve the expression of these proteins. In one form, the claimed protein presents the sequence (SEQ ID N^{o} 2) or (SEQ ID N^{o} 3).

SEQ ID N^{o} 1

X₁X₂X₃X₄X₅SVLTSKIRNFX₆X₇KX₈LELQIPGHTDLLHLIRELAX₉EP
X₁₀GIKYLVESYEEFNQLMHVINFHYDLLRAKLHDMCAHDYCKIPEHLKI
SDKELDMLKKVVLGYRKPLDNIKDDIGKLEX₁₁FITKNKX₁₂TIX₁₃NI
X₁₄X₁₅LIX₁₆X₁₇ENX₁₈KRX₁₉X₂₀X₂₁X₂₂TX₂₃TTNGX₂₄GX₂₅Q
X₂₆X₂₇X₂₈X₂₉X₃₀X₃₁X₃₂X₃₃X_{34G}X₃₅X₃₆X₃₇TGX₃₈X₃₉X₄₀S
X₄₁SSX₄₂TX₄₃SX₄₄GX₄₅GX₄₆TX₄₇X₄₈GX₄₉SX₅₀PAX₅₁AX₅₂X₅₃
SSTNX₅₄X₅₅YX₅₆X₅₇KKX₅₈IYQAX₅₉YNX₆₀IFYTX₆₁QLX₆₂EAQK
LIX₆₃VLEKRVKVLKEHKX₆₄IKX₆₅LLEQVX₆₆X₆₇EKX₆₈KLPX₆₉D
X₇₀X₇₁X₇₂X₇₃TX₇₄LTX₇₅X₇₆X₇₇X₇₈KX₇₉AX₈₀X₈₁KIAX₈₂LE
X₈₃X_{84I}X₈₅AX₈₆AKTVNFDLDGLFTDAEELEYYLREKAKMAGTLIIPE
STKSAGTPGKTVPTLKFTYPH

Where X means:
X_{1, 3, 86}: o N
X_{2, 23, 72, 73}: N o T
X₄: Q o F
X₅: V o P
X₆: V o L
X_{7, 19, 20, 32, 44}: G o S
X₈: S o F
X₉: F o V
X₁₀: N o H
X_{11, 24, 25}: T o A
X₁₂: E o I
X_{13, 69, 81, 83}: S o K
X_{14, 33, 35, 40, 42, 61}: N o S
X₁₅: K o D
X₁₆: S o I
X_{17, 52}: D o A
X_{18, 67}: A o K
X_{21, 78}: Q o H
X_{22, 50}: S o P
X_{26, 74}: N o P
X₂₇: N o A
X₂₈: NGSIAAASSETTQI or is not present.
X₂₉: A o S
X_{30, 41}: A o G
X₃₁: Q o S
X_{34, 36, 38, 46}: T o S
X₃₇: E o S
X₃₉: T, R o S
X₄₃: L o G
X₄₅: A, D o T)
C₄₇: V o G
X₄₈: V o T
X₄₉: T o Q
X_{51, 53}: P o A
X_{54, 57, 63, 79}: A o E
X_{55, 75}: N o D
X_{56, 82}: E o D
X₅₈: I o K
X₅₉: I, V o M
X₆₀: G o T
X_{62, 80}: E o Q
X₆₄: G o D
X₆₅: A o V
X_{66, 68}: K o E
X₇₀: N o Y
X₇₁: T o P
X₇₆: E or its not present
X₇₇: Q o V
X₈₄: Q o K
X₈₅: V o E

The invention also covers the recombinant protein that has previously defined the sequence encoding a fragment of which was modified toward the extreme 5′ by the addition of the genetic code of the methionine(M) amino acid residue followed by the polypeptide ITIFP and 6 histidine (H)amino acid residues. Preferably, the sequence of the recombinant protein that here is claimed is the sequence SEQ ID N^{o} 4.

Other alternative of the invention refers to the protein that has the sequence encoding a fragment which was modified toward the extreme 5′ by the addition of the methionine(M) amino acid residue followed by the polypeptide ITIFP; and in its extreme 3′ where 5 histidine aminoacid (H) residues have been inserted. Preferably, the application refers to the sequence of the recombinant protein that corresponds to the sequence SEQ ID N^{o} 5.

In this order, during development of the invention, two recombinant protein were generated, rPv200L (SEQ ID N^{o} 4) and EcPv200L (SEQ ID N^{o} 5), originated from manipulations to achieve transgenic production in E. coli, improve the hydrophobicity conditions and packing into inclusion bodies, and for their subsequent purification. Specifically, to develop the product rPv200L the genetic codes for the amino acid methionine(M), as an initial codon, a highly hydrophobic short sequence of the ITIFP followed by 5 histidines (H) towards the 5′ gene extreme (SEQ ID N^{o} 6) were added. In the case of EcPv200L a synthetic gene was used (SEQ ID N^{o} 7), with an altered genetic code to make compatible the wild gene using E. coli codon and facilitate the transgenic expression.

Additionally the sequence ITIFP was maintained and genetic code for 6 histidines where added toward 3′ gene extreme, to facilitate the purification process and finally a stop codon was added.. The punctual modifications are shown in the sequence N^{o} 7.

Other invention claimed in the present application is the production process of the EcPv200L recombinant protein. This production process involves 3 sub-processes: cloning, fermentation and purification. During cloning, the synthetic gene is inserted in to the pET-24(a) prokaryotic expression vector and subsequently are transformed in E. coli bacteria BL21 (DE3). In the fermentation sub process, the recombinant clones are adapted to grow in a chemically defined culture and in a bioreactor controlled conditions. Finally, for the purification stage the resulting cellular mass is lyzed in a microfluidizator and after previous centrifugation inclusion bodies are recovered. The last are solubilized with GuHC1 8M in the presence of β-mercaptoetanol and then are submitted to purification by a series of chromatography.

The Chromatography leading to purify and obtain the final product in order are: 1) Affinity chromatography with metal ion nickel (IMAC), 2) size exclusion chromatography in Superedex 300 (SEC-S300); 3) hydrophobic interaction chromatography in butyl resin (HIC-Butyl); 4) ionic exchange chromatography in Q resin (IEX-Q); and 5) size exclusion chromatography (SEC-S75) in Superdex 75. The final product is characterized to determine the size exclusion analytic chromatography profile (SEC-An) and reverse phase (RP-An) HPLC, the endotoxins content and E.coli contaminants and finally the mass spectrometry profile and N-terminal sequencing. The process for obtaining the recombinant protein EcPv200L EcPv200L is novel as it is a unique process designed for this recombinant protein based on its biochemical characteristics and had not been reported in the state of the art. In addition, the process itself does not work with any other recombinant protein and with the protein of invention has the advantage to ensuring a high production without affecting either it stability nor other relevant characteristics for the conservation of antigenic properties of the protein, which benefits that could not be reach if the stages or conditions herein defined are modified. Furthermore is part of the invention the sequences of nucleic acids coding for the Pv200L fragment, as well as the complementary nucleic acid molecules (cDNA) and the variation that could have this DNA molecule due to the genetic code degeneration. Similarly, the claimed invention covers the expression vectors that include the DNA or cDNA previously defined and the transformed cells with those vectors. Inside the vectors used in for this invention are plasmids, phagos, baculovirus and YAC, expressed in prokaryotic systems such as bacteries, and eukaryotic systems such as yeast, plants cells, mammals and insects.

Additionally to previously described inventions, pharmaceutical compositions are also part of the invention especially vaccines for malaria prevention that include the Pv200L subunit or their subfragments both as a synthetic peptide or recombinant protein, DNA or RNA.

Preferably, the invention refers to pharmaceutical compositions previously described, and comprising one or more adjuvants for human use, because it's widely recognized in vaccines formulations to potentiate the immune response, either for specific antibodies induction and/or T helper and/or cytotoxics lymphocytes stimulation

In a complementary way, is objective of the present invention, the formulation of the immunogenic molecules previously mentioned, either individually or combined with adjuvants or combined with other immunogenic molecules formulated in pharmaceutical compositions to be administered in patients with the aim to prevent malaria infections; these molecules can be formulated as pharmaceutical compositions as recombinant protein forms and/or synthetic peptides formulated in different adjuvants for human use and in different proportions.

According to the above mentioned, the current invention also includes the pharmaceutical compositions that comprise immunogenic molecules prior defined with one or more adjuvants selected from a group that includes: Montanide ISA-720, Montanide ISA-51, ASO2 (SBAS2), AS2V, AS1B, MF59, Alum, QS-21, MPL, CpG or microcapsules. These adjuvants have been used with different Plasmodium's antigens and have proven to be safe and able to stimulate both humoral and/cellular immune response.

Additionally, pharmaceutical compositions involves in the claimed invention that comprise the immunogenic molecules previously defined and fragments derived from other Plasmodium stages or from different microorganisms to these one and optionally include different adjuvants for humans use

Preferably, the subunit of the invention may be combined with antigens present in the different phases of the parasite life cycle, either with antigens added to the sequence during the synthesis or added to the pharmaceutical composition, such as the circumsporozoite protein (CS), the adhesion protein related to the trombospondin (TRAP), the Duffy binding protein (DBP), the merozoite surface protein (MSP-1), the Pvs25 protein and the Pvs48/45 protein from the sporogonic cycle among others. These antigens may be used complete or fragments of them produced as synthetic peptides, recombinant proteins or DNA.

To illustrate the information given above, here examples are presented to describe in a detailed form the best way to carry out the characterization of P. vivax Pv200L subunit and its production as a recombinant protein with different molecules of the invention. However, the claimed subject is not limited to those examples. On the contrary, the requested object covers the protein of the present invention or its fragments independently of the process used for it production

### Example 1

### Production Systems of Pv200L as a Recombinant Protein

The Pv200L subunit has been produced as a recombinant protein. During the development process of invention two recombinant prototypes were obtained, rPv200L and EcPv200L. The rPv200L was obtained from a PCR amplified product, that was inserted into a plasmidic vector pRSET-B, which was subsequently cloned in a BL21(DE3)-RIL E. coli bacteria. The rPv200L purification was achieved by a step wise process throughout a manual Nickel column chromatography (IMAC), process facilitated by the addition of histidines toward the N-terminal extreme.

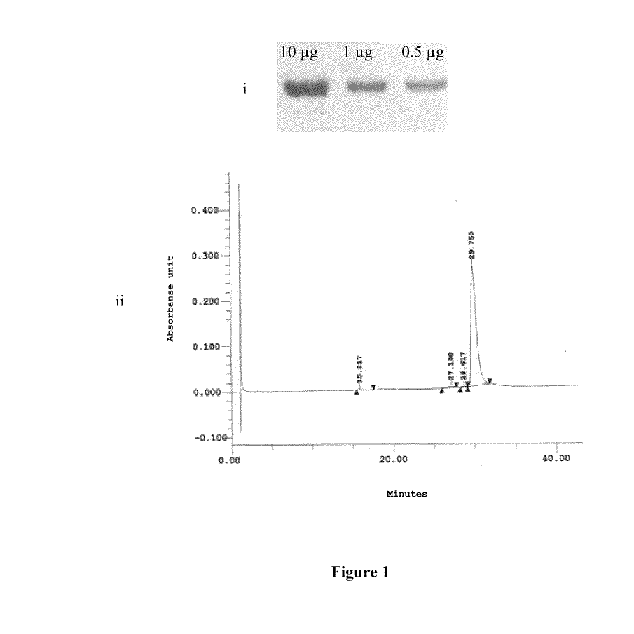

The product obtained can be observed in FIG. 1, in which is shown: (i) acrilamide gel electrophoresis stained with Coomassie Blue of the rPv200L at 10, 1 and 0.5 μg. (ii) chromatographic profile by reverse phase HPLC demonstrating more than 90% of homogeneity in the final product.

Due to promissory results obtained with this unit, we decided to establish and improve the production system to offer a better recombinant Pv200L version denominated as EcPv200L, compatible with standard of good clinical practice.

The main changes in the expression systems are summarize in table 1. The most important are: change in the expression vector, the production of a synthetic gene harmonized to be use with the E. coli codon, the change of the selection marker, the, production of the cellular mass in a bioreactor and in a chemical define culture medium.

TABLE 1
EcPv200L production

| Characteristics | rPv200L | EcPv200L |
|---|---|---|
| Vector | pRSET-B | pET24a(+) |
| Gen source | wild | Armonizad synthetic gen |
| Selection marker | Ampiciline resistance | Kanamycin resistance |
| Host | E. coli | E. coli BL21(DE3) |
| | BL21(DE3)-RIL | |
| Culture medium | Complex | Define |
| Bioreactor | No | Yes |
| Quality standar | Laboratory | Compatible with clinical |
| | | grade |
| Corpuscular Fracción | Soluble | Inclusión bodies |

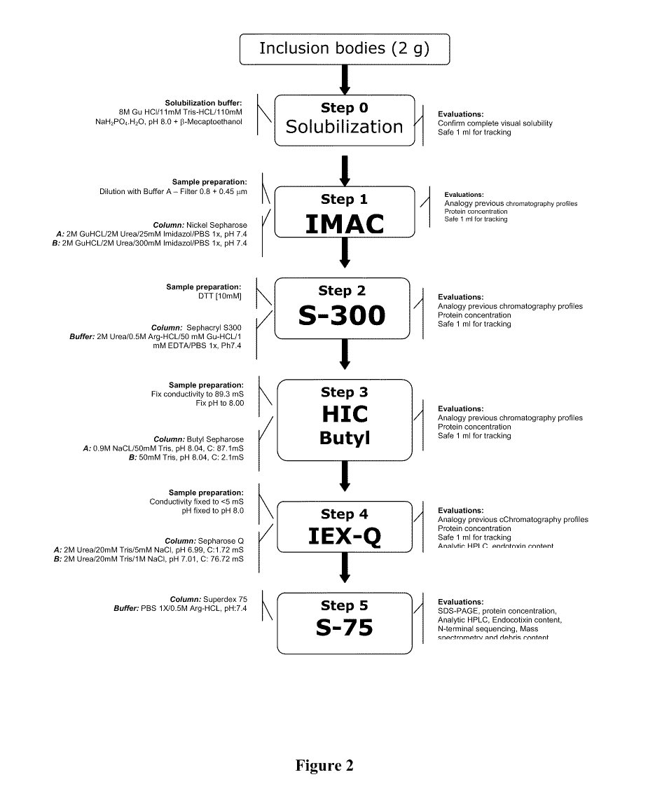

Finally, it was established a purification system with a total of 5 steps which include: affinity chromatography (IMAC), size exclusion chromatography (SEC-s300 and SEC-S75), ionic exchange chromatography (IEX-Q) and hydrophobic interaction (HIC-Butyl). This procedure and its correct sequence are detailed in FIG. 2. The final product was biochemically characterized in terms of: plasmid expression sequence, reverse phase profile and size exclusion chromatography (SEC) by HPLC with hydrodynamic ratio analysis, N-terminal sequencing, mass spectrometry profile (MS), endotoxins content and E. coli contaminants.

The process described above has a global purification efficiency of 20% and is easily scale up and transferable. The obtain product, presents endotoxins levels below 100 UE/dose and almost undetectable E. coli contaminants (<0.1%), compatible with regulations of the FDA and USP agencies. Consequently, the process herein defined ensures a better protein expression, a higher productivity and an optimal protein quality to be used in vaccines production.

### Example 2

### Seroepidemiology Assays of Pv200L Subunit

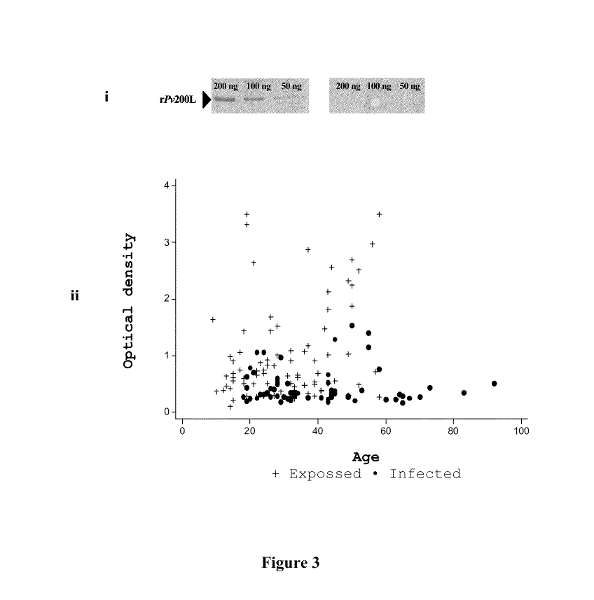

The Pv200L subunit is highly recognized by subjects of endemic areas who have been infected and/or exposed to P. vivax infection. FIG. 3 shows IgG antibodies levels against rPv200L found during a seroepidemiologycal study carried out in the colombian Pacific Coast.

Sera from P. vivax-infected individuals recognized specifically the rPv200L recombinant protein by immunoblot (FIG. 3i, left), while control subjects, who have never visited or been exposed in endemic areas, do not recognize the rPv200L (FIG. 3i, right). Distribution of optical density values according to age (FIG. 3i) shows that there is a tendency to develop higher antibody titers at 2 and 5 decade of life, this phenomenon is more remarkable among exposed (•) than infected individuals (+).

TABLE 2
Seroepidemiology of Pv200L

| | | Positive % IgG | | |
|---|---|---|---|---|
| | | (IC exacto | OD Average | Antibodies |
| Group | η | 95%) | (IC 95%) | titers |
| Infected | 81 | 72.8 | 1.015 | 10³-10⁵ |
| | | (61.8-82.1) | (0.828-1.203) | |
| Exposed | 69 | 52.2 | 0.464 | 10²-10⁴ |
| | | (39.8-64.3) | (0.363-0.506) | |
| Control | 44 | 6.8 | 0.264 | — |
| | | (1.40-18.1) | (0.246-0.368) | |

Table 2 shows the level of recognition of the Pv200L subunit by subjects from an endemic area in terms of percentage of responders with IgG positive reaction against rPv200L and the strength of such reaction expressed as mean of the optical density.

The recombinant protein EcPv200L was analyzed by seroepidemiologycal studies of sera from subjects of Brazilian endemic areas, obtaining a positive IgG recognition of the subunit in more than 90% of 4 different endemic areas evaluated.

### Example 3

### Immunogenicity of rPv200L in BALB/c Mice

BALB/c mice were immunized with 50 μg of the rPv200L under a regimen of three intraperitoneally immunizations. Levels of specific IgG antibodies against rPv200L after the third immunization of the protein emulsified in Freund adjuvant is shown in

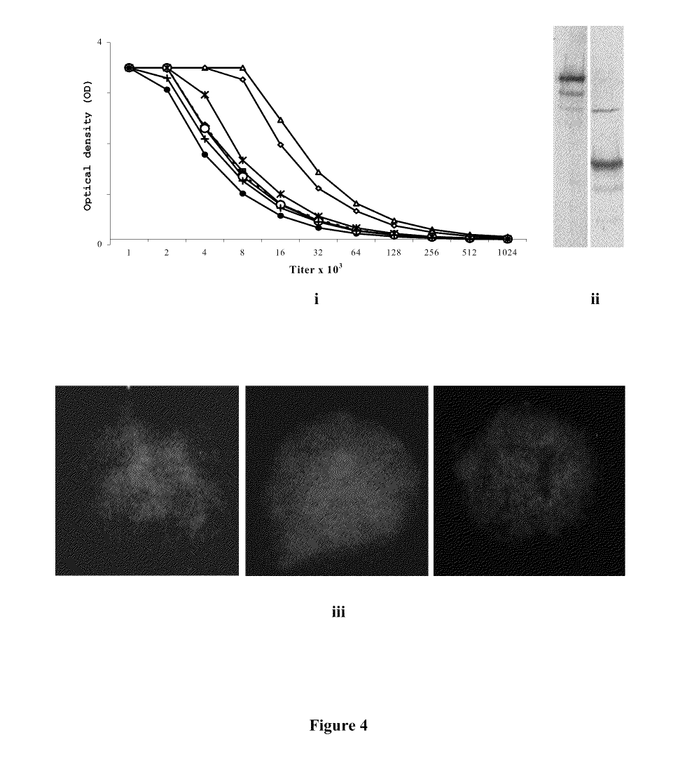

FIG. 4. After last immunization, antibody titer of IgG type anti-rPv200L reach levels over 1×10⁷ dilutions (FIG. 4i). Such antibodies are capable to recognize the immunogen rPv200L (4ii, left) and its P. falciparum homologous rPf190L (4ii, right). Finally, antibodies induced by immunization with rPv200L in BALB/c mice were able to recognize the native protein on P. vivax schizont (400iii).

### Example 4

### Immunogenicity and Protective Efficacy of rPv200L in Primates of the Aotus Genus

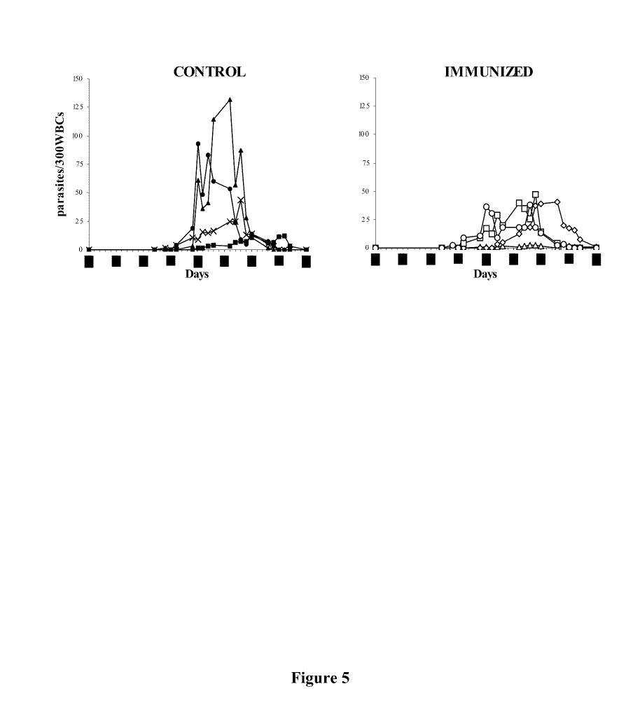

The immunization of Aotus lemurinus griseimembra monkeys with 3 subcutaneous doses of 100 μg of rPv200L emulsified in freund adjuvant, induce a strong immune response in terms of IgG antibodies that protects against challenge with P. vivax blood forms (Salvador I). Immunized monkeys are partially protected with rPv200L by controlling parasitemia and its progress to severe anemia. FIG. 5i shows that the peaks of parasitemia curves (parasites/300 WBCs) reached by the control group (left) are higher than the immunized group (right).

Table 3 summarized the parameters determined in the protective efficacy of a pilot study in Aotus monkeys, demonstrating that cumulative parasitemia (CP) and the area under the parasitemia curve (AUC) is lower in the immunized group. Similarly, data of the humoral immune response is shown. Only animals from control group had to be treated to control parasitemia before getting severe anemia, supporting the evidence of immune response against the severity of the disease.

TABLE 3
Protective Efficacy of rPv200L in Aotus primates

| | IgG tittle | | CP | | AUC | |
|---|---|---|---|---|---|---|
| Group | ELISA | IFAT | Pcl | Ptx | Pcl | Ptx |
| Control | <1000 | Negative | 575 | 562 | 840 | 800 |
| | <1000 | Negative | 201 | 179 | 280 | 232 |
| | <1000 | Negative | 90 | 36 | 121 | 40 |
| | <1000 | Negative | 425 | 400 | 586 | 537 |
| Immunized | 2 × 10⁷ | 2,200 | 19 | 11 | 25 | 13 |
| | 2 × 10⁷ | 2,200 | 235 | 95 | 339 | 93 |
| | 2 × 10⁷ | 2,200 | 259 | 240 | 353 | 292 |
| | 2 × 10⁷ | 2,200 | 231 | 210 | 312 | 266 |

ELISA = IgG antibody titters against rPv200L before challenge;
IFAT = IgG antibody titers against P. vivax schizonts before challenge;
CP = Cumulative Parasitemia (Σ of parasitemia/300 white blood cells each year);
Pcl = Parasitemia clearance period;
Ptx = prepatent period (for all group);
AUC = Area under de Curve{parasites/day}.

1. A protein that is characterized in that it comprises the sequence:
X₁X₂X₃X₄X₅SVLTSKIRNFX₆X₇KX₈LELQIPGHTDLLHLIRELAX₉EP
X₁₀GIKYLVESYEEFNQLMHVINFHYDLLRAKLHDMCAHDYCKIPEHLKI
SDKELDMLKKVVLGYRKPLDNIKDDIGKLEX₁₁FITKNKX₁₂TIX₁₃NI
X₁₄X₁₅LIX₁₆X₁₇ENX₁₈KRX₁₉X₂₀X₂₁X₂₂TX₂₃TTNGX₂₄GX₂₅Q
X₂₆X₂₇X₂₈X₂₉X₃₀X₃₁X₃₂X₃₃X₃₄GX₃₅X₃₆X₃₇TGX₃₈X₃₉X₄₀S
X₄₁SSX₄₂TX₄₃SX₄₄GX₄₅GX₄₆TX₄₇X₄₈GX₄₉SX₅₀PAX₅₁AX₅₂
X₅₃SSTNX₅₄X₅₅YX₅₆X_{S7}KKX₅₈IYQAX₅₉YNX₆₀IFYTX₆₁QlX₆₂E
AQKLIX₆₃VLEKRVKVLKEHKX₆₄IKX₆₅LLEQVX₆₆X₆₇EKX₆₈KLP
X₆₉DX₇₀X₇₁X₇₂X₇₃TX₇₄LTX₇₅X₇₆X₇₇X₇₈KX₇₉AX₈₀X₈₁KIA
X₈₂LEX₈₃X₈₄IX₈₅AX₈₆AKTVNFDLDGLFTDAEELEYYLREKAKMAGT
LIIPESTKSAGTPGKTVPTLKETYPH
Where X means:
X_{1, 3, 86}: I or N
X_{2, 23, 72, 73}: N or T
X₄: Q or F
X₅: V or P
X₆: V or L
X_{7, 19, 20, 32, 44}: G or S
X₈: S or F
X₉: F or V
X₁₀: N or H
X_{11, 24, 25}: T or A
X₁₂: E or I
X_{13, 69, 81, 83}: S Or K
X_{14, 33, 35, 40, 42, 61}: N or S
X₁₅: K or D
X₁₆: S or I
X_{17, 52}: D or A
X_{18, 67}: A or K
X_{21, 78}: Q or H
X_{22, 50}: S or P
X_{26, 74}: N or P
X₂₇: N or A
X₂₈: NGSIAAASSETTQI or it is not present.
X₂₉: A or S
X_{30, 41}: A or G
X₃₁: Q or S
X_{34, 36, 38, 46}: T or S
X₃₇: E or S
X₃₉: T, R or S
X₄₃: L or G
X₄₅: A, D or T
X₄₇: V or G
X₄₈: V or T
X₄₉: T or Q
X_{51, 53}: P or A
X_{54, 57, 63, 79}: A or E
X_{55, 75}: N or D
X_{56, 82}: E or D
X₅₈: I or K
X₅₉: I, V or M
X₆₀: G or T
X_{62, 80}: E or Q
X₆₄: G or D
X₆₅: A or V
X_{66, 68}: K or E
X₇₀: N or Y
X₇₁: T or P
X₇₆: E or it is not present
X₇₇: Q or V
X₈₄: Q or K
X₈₅: V or E.

2. The protein, according to claim 1, wherein it comprises the aminoacid sequence defined in SEQ ID N^{o} 2 or SEQ ID N^{o} 3.

3. The protein, according to claim 1, wherein it is a recombinant protein or a synthetic peptide.

4. The recombinant protein, according to claim 3, wherein it additionally has a methionine aminoacid (M) followed by 6 histidine aminoacids and the ITIFP peptide toward N-terminal extreme.

5. The recombinant protein according to claim 4, wherein it comprises the sequence SEQ ID N^{o} 4.

6. The recombinant protein according to claim 3, wherein it additionally has a methionine aminoacid (M) followed by ITIFP peptide toward N-terminal extreme, and five histidine aminoacids towards the C-terminal extreme.

7. The recombinant protein according to claim 6, wherein it comprises the sequence SEQ ID N^{o} 5.

8. A nucleic acid molecule wherein it comprises a nucleotide sequence that encodes a protein according to claim 1.

9. The nucleic acid molecule, according to claim 8, wherein it is a DNA, RNA or cDNA molecule.

10. The nucleic acid molecule, according to claim 9, wherein it comprises the polynucleotide sequence identified as SEQ ID N^{o} 6 or SEQ ID N^{o} 7.

11. An expression vector wherein it comprises the nucleic acid molecule according to claim 8, and wherein the polynucleotide is the sequence identified as SEQ ID N^{o} 6 or the sequence identified as SEQ ID N^{o} 7.

12. The expression vector, according to claim 11, wherein it is a plasmid or a phagus.

13. A recombinant cell wherein it comprises the expression vector according to claim 11.

14. A pharmaceutical composition for malaria prevention wherein it comprises a protein according to claim 1.

15. A pharmaceutical composition for malaria prevention wherein it comprises a nucleic acid molecule according to claim 8.

16. A pharmaceutical composition for malaria prevention wherein it comprises the vector according to claim 11.

17. A pharmaceutical composition for malaria prevention wherein it comprises the recombinant cell according to claim 13.

18. A vaccine for malaria prevention wherein it comprises the protein according to claim 1.

19. A vaccine for malaria prevention wherein it comprises the nucleic acid molecule according to claim 8.

20. A vaccine for malaria prevention wherein it comprises the vector according to claim 11.

21. A vaccine for malaria prevention wherein it comprises the recombinant cell of claim 13.

22. The vaccine according to claim 17, wherein it comprises in addition one or more adjuvants for human use.

23. The vaccine according to claim 22 wherein the adjuvant is selected from the group consisting of Montanide ISA-720, Montanide ISA-51, ASO2 (SBAS2), AS2V, AS1B, MF59, Alum, QS-21, MPL, CpG or microcapsules.

24. The vaccine, according to claim 22, wherein it comprises in addition antigens derived from other Plasmodium stages or from different microorganisms.

25. The vaccine according to claim 24 wherein the antigens derived from other Plasmodium stages or from different microorganisms are selected from a group that consists of the adhesion protein related with the trombospondina (TRAP), the Duffy binding protein (DBP), the merozoite surface protein (MSP-1), Pvs25 protein and Pv48/45, among others.

26. The vaccine according to claim 25 wherein the antigens are both complete proteins or protein fragments produced as synthetic peptides, recombinant proteins or DNA vaccines.

27. A process to produce the recombinant protein of claim 1 wherein it comprises the following steps:
a) cloning of the synthetic gen encoding the aminoacids sequence defined in SEQ ID 1 in the prokaryotic expression vector pET-24(a) and transformation of bacteria E. coli BL21 (DE3) with said vector;
b) fermentation of the recombinant clones in a chemically defined culture medium in a bioreactor controlled conditions; and
c) cellular mass purification by lysis in a microfluidizator followed by centrifugation to recover inclusion bodies, which are solubilized with GuHC1 8M in the presence of β-mercaptoetanol and subsequently subjected to purification by a series of chromatographies.

28. The process, according to claim 27, wherein preferably the order of the chromatographies in Step c are:
ion metallic nickel affinity chromatography (IMAC)
superdex (SEC-S300) size exclusion chromatography
hydrophobic interaction chromatography in butyl resin (HIC-Butyl)
ionic exchange chromatography in Q resin; and
superdex 75 (SEC-575) Size exclusion chromatography
